(19) United States
(12) Patent Application Publication
(10) Pub. No.: US 2025/0352058 A1
(43) Pub. Date: Nov. 20, 2025

(54) GENERATING AND EVALUATING TWO- AND THREE-DIMENSIONAL IMAGES OF THE INTERIOR OF AN EYE

(71) Applicant: Alcon Inc., Fribourg (CH)

(72) Inventor: Utkarsh SHARMA, Solon, OH (US)

(21) Appl. No.: 19/286,068

(22) Filed: Jul. 30, 2025

Related U.S. Application Data
(63) Continuation of application No. 17/938,187, filed on Oct. 5, 2022, now Pat. No. 12,390,102.
(60) Provisional application No. 63/281,453, filed on Nov. 19, 2021.

(51) Int. Cl. A61B 3/10; A61B 3/00; A61F 9/008; G02B 26/10; G06T 7/13; G06T 7/60; G06T 7/73

(52) U.S. Cl. CPC A61B 3/102; A61B 3/0058; A61F 9/008; G02B 26/101; G06T 7/13; G06T 7/60; G06T 7/73; G06T 2207/10101; G06T 2207/20216; G06T 2207/30041

(57) ABSTRACT

In certain embodiments, an ophthalmic laser surgical system for imaging and treating a target in an eye includes an optical coherence tomography (OCT) device that: directs an imaging beam towards the eye; generates three-dimensional (3D) image data from the imaging beam reflected from the eye; and generates two-dimensional (2D) enface images from the 3D image data. The 2D enface images include a target enface image imaging the target in the eye and a retinal enface image imaging a shadow cast by the target onto the retina. An xy-scanner directs the imaging beam along an imaging beam path towards the eye, and directs a laser beam from the laser device along a laser beam path aligned with the imaging beam path towards the eye. A computer compares the target of the target enface image and the shadow of the retinal enface image to confirm the presence of the target.

### CROSS-REFERENCE TO RELATED APPLICATIONS

This application is a continuation of U.S. patent application Ser. No. 17/938,187, filed Oct. 5, 2022, which claims benefit of and priority to U.S. Provisional Application No. 63/281,453, filed Nov. 19, 2021, the entire contents of each being incorporated herein by reference.

### TECHNICAL FIELD

The present disclosure relates generally to ophthalmic surgical systems, and more particularly to generating and evaluating two- and three-dimensional images of the interior of an eye.

### BACKGROUND

Laser vitreolysis uses laser beams to treat vitreous floaters and other retinal diseases. Precise delivery of a laser beam to the target is important to avoid damaging healthy tissue and ensure ocular safety. Accordingly, imaging systems should provide sufficiently clear images of targets. However, known imaging systems are not satisfactory in certain situations.

### BRIEF SUMMARY

In certain embodiments, an ophthalmic laser surgical system for imaging and treating a target in an eye includes an optical coherence tomography (OCT) device, a laser device, an xy-scanner, and a computer. The eye has an eye axis that defines a z-axis, which defines xy-planes within the eye. The OCT device directs an imaging beam along an imaging beam path towards the eye; receives the imaging beam reflected from the eye; generates three-dimensional (3D) image data from the reflected imaging beam; and generates two-dimensional (2D) enface images from the 3D image data. A 2D enface image images an xy-plane within the eye. The 2D enface images include a target enface image imaging the target in the eye and a retinal enface image imaging a retina of the eye. The retinal enface image shows a shadow cast by the target onto the retina. The laser device directs a laser beam along a laser beam path towards the target. The xy-scanner receives the imaging beam from the imaging system and directs the imaging beam along the imaging beam path towards the eye; and receives the laser beam from the laser device and directs the laser beam along the laser beam path aligned with the imaging beam path towards the eye. The computer compares the target of the target enface image and the shadow of the retinal enface image to confirm the presence of the target.

Embodiments may include none, one, some, or all of the following features:
The target comprises a vitreous eye floater.
The OCT device generates the two-dimensional (2D) enface images from the 3D image data by: taking a slice of the 3D image data; and summing data of the slice to yield a 2D enface image.
The OCT device generates the two-dimensional (2D) enface images from the 3D image data by: taking a slice of the 3D image data; and averaging data of the slice to yield a 2D enface image.
The OCT device generates the two-dimensional (2D) enface images from the 3D image data by: taking a slice of the 3D image data; and projecting data of the slice to yield a 2D enface image.
The computer performs image processing on the target enface image to determine a feature of the target. The computer may perform image processing on the target enface image to identify an outline of the target, and determine a size of the target from the outline of the target. The computer may perform image processing on the target enface image to identify an outline of the target, and determine a shape of the target from the outline of the target.
The computer tracks the target according to the target enface image imaging the target.
The computer tracks the target by tracking the shadow cast by the target according to the retinal enface image.
The computer overlays an outline of the target onto the target enface image.
The computer overlays a no-fire zone onto the target enface image.
The OCT device generates three-dimensional (3D) images from the 3D image data.

In certain embodiments, a method images and treats a target in an eye. The eye has an eye axis that defines a z-axis, which defines xy-planes within the eye. The method includes: directing, by an optical coherence tomography (OCT) device, an imaging beam along an imaging beam path towards the eye; receiving the imaging beam reflected from the eye; generating three-dimensional (3D) image data from the reflected imaging beam; and generating two-dimensional (2D) enface images from the 3D image data. A 2D enface image images an xy-plane within the eye. The 2D enface images include a target enface image imaging the target in the eye and a retinal enface image imaging a retina of the eye. The retinal enface image shows a shadow cast by the target onto the retina. The method further includes: directing, by a laser device, a laser beam along a laser beam path towards the target; receiving, by an xy-scanner, the imaging beam from the imaging system and direct the imaging beam along the imaging beam path towards the eye; receiving, by the xy-scanner, the laser beam from the laser device and direct the laser beam along the laser beam path aligned with the imaging beam path towards the eye; and comparing, by a computer, the target of the target enface image and the shadow of the retinal enface image to confirm the presence of the target.

Embodiments may include none, one, some, or all of the following features:
The generating the two-dimensional (2D) enface images from the 3D image data includes: taking a slice of the 3D image data; and summing, averaging, or projecting data of the slice to yield a 2D enface image.
The method further includes performing, by the computer, image processing on the target enface image to determine a feature of the target. The computer may perform image processing on the target enface image to identify an outline of the target, and determine a size of the target from the outline of the target. The computer may perform image processing on the target enface image to identify an outline of the target, and determine a shape of the target from the outline of the target.
The method further includes tracking, by the computer, the target according to the target enface image imaging the target.
The method further includes tracking, by the computer, the target by tracking the shadow cast by the target according to the retinal enface image.
The method further includes overlaying, by the computer, an outline of the target or a no-fire zone onto the target enface image.
The method further includes generating, by the OCT device, a plurality of three-dimensional (3D) images from the 3D image data.

In certain embodiments, an ophthalmic laser surgical system for imaging and treating a target in an eye includes an optical coherence tomography (OCT) device, a laser device, an xy-scanner, and a computer. The eye has an eye axis that defines a z-axis, which defines xy-planes within the eye. The target is a vitreous eye floater. The OCT device directs an imaging beam along an imaging beam path towards the eye; receives the imaging beam reflected from the eye; generates three-dimensional (3D) image data from the reflected imaging beam; and generates two-dimensional (2D) enface images from the 3D image data. The OCT device generates the 2D enface images from the 3D image data by taking a slice of the 3D image data and summing, averaging, or projecting data of the slice to yield a 2D enface image. A 2D enface image images an xy-plane within the eye. The 2D enface images include a target enface image imaging the target in the eye and a retinal enface image imaging a retina of the eye. The retinal enface image shows a shadow cast by the target onto the retina. The laser device directs a laser beam along a laser beam path towards the target. The xy-scanner receives the imaging beam from the imaging system and directs the imaging beam along the imaging beam path towards the eye; and receives the laser beam from the laser device and directs the laser beam along the laser beam path aligned with the imaging beam path towards the eye. The computer performs the following: compares the target of the target enface image and the shadow of the retinal enface image to confirm the presence of the target; performs image processing on the target enface image to determine a feature of the target by performing image processing on the target enface image to identify an outline of the target and determining a size of the target from the outline of the target, and by performing image processing on the target enface image to identify an outline of the target and determining a shape of the target from the outline of the target; tracks the target according to the target enface image imaging the target; tracks the target by tracking the shadow cast by the target according to the retinal enface image; overlays an outline of the target onto the target enface image; overlays a no-fire zone onto the target enface image; and generates a plurality of three-dimensional (3D) images from the 3D image data.

### BRIEF DESCRIPTION OF THE DRAWINGS

FIG. 1 illustrates an example of an ophthalmic surgical system that can image and treat a target in an eye, according to certain embodiments;

FIGS. 2A and 2B illustrate examples of two-dimensional (2D) enface images that may be generated by the OCT device of the system of FIG. 1;

FIG. 3 illustrates an example of a three-dimensional (3D) image that may be generated by the OCT device of the system of FIG. 1; and

FIG. 4 illustrates an example of a method for imaging and treating a target in the vitreous of an eye, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Known surgical systems include imaging systems, such as scanning laser ophthalmoscope (SLO) and optical coherence tomography (OCT) devices, to provides images of targets or their shadows. An SLO device provides two-dimensional (2D) enface images of a target or its shadow on the retina, and an OCT device provides three-dimensional (3D) images of the target. However, these known surgical systems have disadvantages. The two different imaging technologies, SLO and OCT, add expense and complexity to the system. In addition, the known surgical systems make it difficult to determine whether the image shows a target shadow or, e.g., a lens opacity, vignetting, retinal pathology, or imaging artifact.

Accordingly, embodiments of the surgical systems described herein include an OCT device that gathers 3D OCT data and generates both 3D images and 2D enface images from the data. A 2D enface image can be generated from a “slice” of the 3D OCT data between two surfaces, e.g., two xy-planes or layers of the tissue of the eye. A spatial mapping of features between the surfaces yields a 2D enface image.

Certain embodiments may offer several advantages. The surgical systems can use the 2D enface images to determine the presence, size, shape, and/or location of the target. For example, a 2D image of the target can be compared with a 2D image of its shadow to confirm the presence of the target. As another advantage, the surgical systems use only one device (the OCT device) instead of two devices (the OCT and SLO devices) to provide 3D images and 2D enface images, which reduces the cost and complexity of certain embodiments.

As yet another advantage, the surgical systems co-register the OCT and laser devices such that the laser beam can be precisely directed to the target using a 2D enface image. The OCT and laser devices share a beam path through the same optical elements, including an xy-scanner, and through the eye. If the OCT and laser beams are aligned prior to the shared optical elements, the beams are automatically aligned at the target location in the eye, allowing for precise image-guided beam targeting.

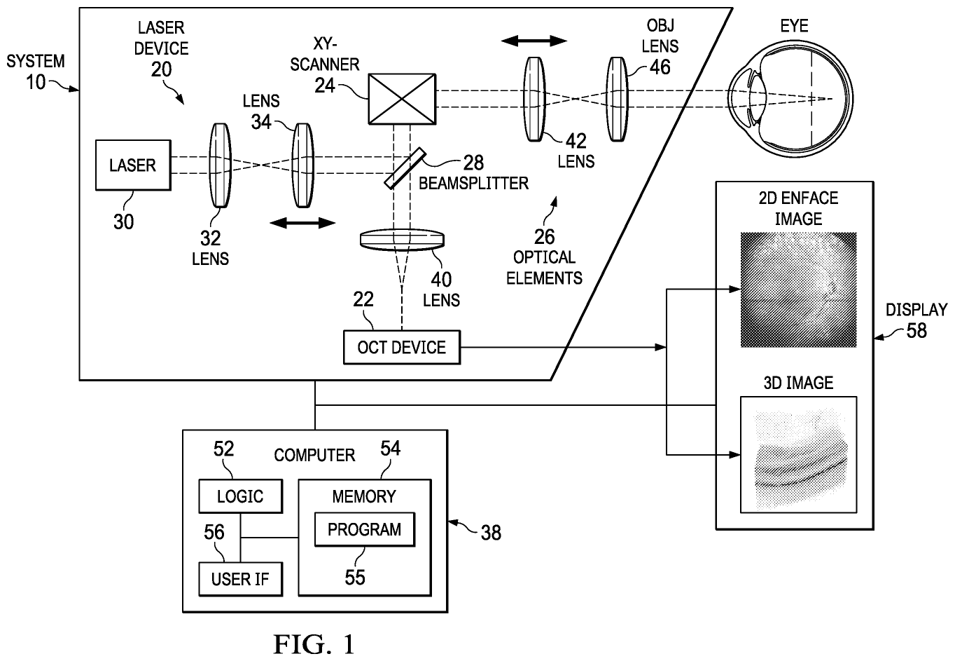

FIG. 1 illustrates an example of an ophthalmic surgical system 10 that can image and treat a target in an eye, according to certain embodiments. In the example, the target is a vitreous eye floater in the vitreous of the eye. In the example, an axis of the eye (e.g., visual or optical) defines a z-axis, which in turn define x- and y-axes orthogonal to the z-axis. X- and y-axes define xy-planes within the eye. X-, y-, and z-directions and locations are relative to the x-, y-, and z-axes, respectively.

In the example, system 10 includes a treatment system (which comprises a laser device 20), an imaging system (which comprises an optical coherence tomography (OCT) device 22), an xy-scanner 24, optical elements 26, and a computer 38, coupled as shown. Laser device 20 includes a laser 30 and lenses 32, 24, coupled as shown. Optical elements includes beamsplitter (e.g., a dichroic mirror (DM)) 28 and lenses 32, 34, 40, 42, and 46, coupled as shown. Computer 38 includes logic 52, memory 54 (which stores a computer program 55), a user interface (IF) 56, and a display 58, coupled as shown.

As an example of operation, OCT device 22 directs an imaging beam along an imaging beam path towards the eye, receives the imaging beam reflected from the eye, generates three-dimensional (3D) image data from the reflected imaging beam, and generates two-dimensional (2D) enface images from the 3D image data. A 2D enface image images an xy-plane within the eye. For example, a target enface image images a target in the eye. Laser device 20 directs a laser beam along a laser beam path towards the target. OCT device 22 and laser device 20 share the same xy-scanner 24, which allows for precise aiming of the laser beam using the imaging. That is, xy-scanner 24 receives the imaging beam from the imaging system and directs the imaging beam along the imaging beam path towards the target, and receives the laser beam from the laser device and directs the laser beam along the laser beam path aligned with the imaging beam path towards the target. In certain embodiments, OCT device 22 combines 2D enface images to generate a three-dimensional (3D) image, where the 3D image images a volume within the eye.

Turning to the treatment system, laser 30 of laser device 22 generates a laser beam with any suitable wavelength, e.g., in a range from 400 nm to 2000 nm. Laser device 22 delivers laser pulses at any suitable repetition rates ranging from, but not limited to, 1 hertz (Hz) to several hundreds of kilohertz (kHz). A laser pulse may have any suitable pulse duration (e.g., ranging from, but not limited to, a nanosecond (ns) to 20 femtoseconds (fs)), any suitable pulse energy (e.g., 1 microjoule (μJ) to 10 millijoule (mJ)), and a focal point of any suitable size (e.g., ranging from 3 to 20 microns (μm), such as 7 μm). Lenses 32 and 34 are used to adjust the focus position of the laser beam within tissue, such as eye tissue.

Turning to the imaging system, OCT device 22 generates 3D images and 2D enface images of the interior of the eye from the imaging beam reflected from the eye. A 2D enface image may be regarded as a pseudo-SLO image, as OCT device 22 can generate 2D enface images that are very similar to SLO images. OCT device 22 may be any suitable device that utilizes optical coherence tomography to generate images, e.g., a swept-source OCT (SS-OCT), line-field OCT, full-field OCT, or spectral-domain OCT (SD-OCT) device.

In certain embodiments, OCT device 22 generates the 2D and 3D images from 3D image data determined from the reflected imaging beam. OCT device 22 performs a series of A-scans (i.e., scans in the z-direction), combines the A-scans to form B-scans, and combines the B-scans to yield 3D image data, which can be used to generate a 3D image. To generate a 2D enface image, OCT device 22 takes a slice of the 3D image data that is generally orthogonal to the z-axis. The slice is bounded by two non-intersecting surfaces. The surfaces may represent, e.g., xy-planes or layers of eye tissue. The data in the slice is processed (e.g., averaged, summed, projected) to yield a 2D enface image. For example, for each point (x, y), the values of the image data at point (x, y) are averaged (or summed, projected, or otherwise processed) to yield the value for point (x, y) of the enface image.

In other embodiments, OCT device 22 generates the 2D enface images directly from the A-scans. OCT device 22 generates an A-scan, which a value for an xy-point of an xy-plane. Multiple A-scans yield values for multiple points of an xy-plane, which can be used to generate a 2D enface image at the xy-plane.

Xy-scanner 36 scans treatment and imaging beams transversely in xy-directions. Examples of scanners include a galvo scanner (e.g., a pair of galvanometrically-actuated scanner mirrors that can be tilted about mutually perpendicular axes), an electro-optical scanner (e.g., an electro-optical crystal scanner) that can electro-optically steer the beam, or an acousto-optical scanner (e.g., an acousto-optical crystal scanner) that can acousto-optically steer the beam.

OCT device 22 and laser device 20 share xy-scanner 24, allowing for co-registration between the OCT imaging and treatment beams. That is, xy-scanner 24 receives the imaging beam from the imaging system and directs the imaging beam along the imaging beam path towards the target, and receives the laser beam from the laser device and directs the laser beam along the laser beam path co-aligned with the imaging beam path towards the target. The OCT imaging and treatment beams share the same path through the optics of the system and the eye, so are affected by the same optical properties and distortions along the beam path. Thus, if the imaging and treatment beams are aligned prior to xy-scanner 24, they are automatically aligned at the target location. This enables accurate and precise delivery of the laser beam to the target location identified using OCT images.

Optical elements includes beamsplitter (such as a dichroic mirror (DM)) 28 and lenses 32, 34, 40, 42, and 46, coupled as shown. In general, an optical element can act on (e.g., transmit, reflect, refract, diffract, collimate, condition, shape, focus, modulate, and/or otherwise act on) a laser beam. Examples of optical elements include a lens, prism, mirror, diffractive optical element (DOE), holographic optical element (HOE), and spatial light modulator (SLM). In the example, lens 40 collimates beams to and from beamsplitter 28. Beamsplitter 28 directs beams from OCT device 22 and laser device 20 to xy-scanner 24 and directs beam reflected from the eye back to OCT device 22. Beamsplitter 28 may comprise any suitable beam splitter that can combine beams or separate one beam into multiple beams. For example, a dichroic mirror can combine or split beams of different wavelengths, depending on the configuration. Lenses 32 and 34 collimates the beam from laser 30. Lens 42 and objective lens 46 collimate and focus beams at the eye.

Computer 38 sends instructions to the OCT device and the laser device. Computer 38 may utilize computer programs 55 to perform operations. Examples of computer programs 55 include target imaging, target tracking, image processing, and target evaluation.

In certain embodiments, computer 26 uses an image processing program 55 to perform image processing on an image, e.g., analyze the digital information of the image to extract information from the image. In certain embodiments, computer 26 performs image processing to analyze an image of a target or a target's shadow (i.e., “target shadow”) to obtain information about the target. Localized opacities in the vitreous, such as floaters, can affect vision quality when they are in the path of light and cast a shadow onto the retina. Hence, the target shadow can provide useful information about clinical significance of the floater or other opacity. Moreover, target shadows may yield higher contrast, clearer images than the targets themselves. Accordingly, images of a target shadow may be easier to analyze to evaluate, e.g., the location, size, or density of the target. In addition, it may be easier to track the target shadow to determine the location of the target.

In the embodiments, computer 26 may analyze the target and/or target shadow in any suitable manner. For example, computer 26 may detect a brighter or darker shape in an image (using, e.g., edge detection or pixel analysis) to detect the target or the target shadow. As another example, program 54 may identify an outline of the target, and determine a size and/or shape of the target from the outline. As another example, program 54 may detect the darkness of the target shadow, i.e., how dark the shadow is. In general, a thicker and/or denser target may yield a darker shadow. Similarly, a target closer to the retina may yield a darker shadow. Accordingly, program 54 may analyze the target and/or target shadow to determine clinically relevant information about the target.

In certain embodiments, computer 38 performs image processing to confirm the presence of the target. OCT device 22 generates a target enface image that shows a target candidate and a retinal enface image that shows a shadow. Computer 38 compares the target candidate and the shadow to confirm the presence of the target. For example, computer 38 may align the images to determine that the target casts the shadow. This may rule out shadows caused by, e.g., a lens opacity, vignetting, retinal pathology, or other imaging artifacts. In certain embodiments, computer 38 performs image processing to generate 2D and 3D images, as described above.

In certain embodiments, computer 38 uses a tracking program to track and/or predict the movement of a target. In some situations, the enface image of the target itself may be clearer than the image of the shadow of the target, so computer 38 may track the target using the target enface image rather than the retinal enface image with the target shadow. In other situations, the retinal enface image with the target shadow may be more appropriate, so computer 38 may track the target using the target shadow. The tracking program may predict the movement of the target and send to laser device 20 the location of where the target is predicted to be when the laser beam reaches the target. The images of the target may be used to acquire the target (e.g., determine the fingerprint of a floater) and fire the laser beam at the target.

In certain embodiments, computer 38 generates image overlays to superimpose over images of the eye. Examples of image overlays include an outline of the target, a no-fire zone indicating where the laser should not be fired (such as the foveal region), information describing the target (e.g., target size, shape, and/or density) or the eye, or other suitable overlay that enhances the image. Examples of eye images over which an image overlay may be superimposed include a retinal enface image, target enface image, real time video of the eye, or other suitable image of the eye. For example, computer 38 may superimpose an outline of the target onto a retinal enface image, a no-fire zone onto a target enface image, or the target size, shape, and/or density onto a real time video of the eye.

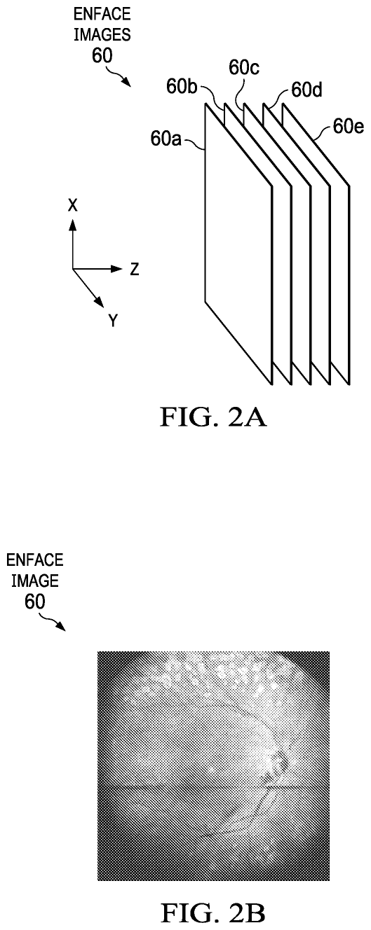

FIGS. 2A and 2B illustrate examples of two-dimensional (2D) enface images 60 that may be generated by OCT device 22 of system 10 of FIG. 1. FIG. 2A shows examples of enface images 60 (60a to 60e). In the examples, each image is located at an xy-plane within the eye, and each xy-plane is located at a different z-location. Enface images 60 include, e.g., a target enface image that images a target in the eye and a retinal enface image that images the retina of the eye. The retinal enface image may also show the shadow cast by the target onto the retina. FIG. 2B shows an example of an enface image 60 generated at the retina of the eye.

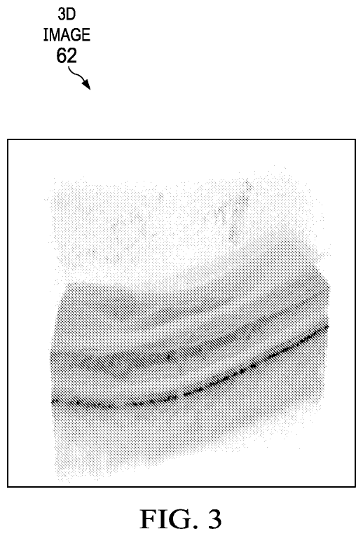

FIG. 3 illustrates an example of a three-dimensional (3D) image 62 that may be generated by OCT device 22 of system 10 of FIG. 1 from 3D OCT image data. In the example, 3D image 62 images a volume within the eye. The volume image may show the target and/or retina.

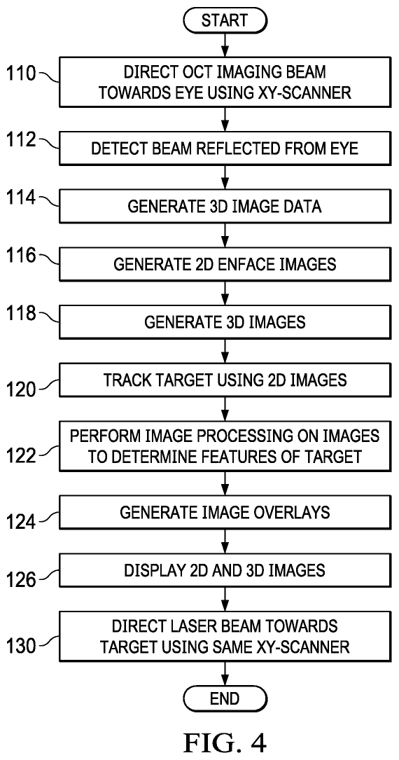

FIG. 4 illustrates an example of a method for imaging and treating a target in the vitreous of an eye, according to certain embodiments. The method starts at step 110, where the OCT device directs an OCT imaging beam towards the eye via an xy-scanner. The OCT device detects the reflected OCT imaging beam at step 112. The OCT device generates 3D image data from the reflected beam at step 114. The image data is generated from multiple A-scans and B-scans.

The OCT device generates 2D enface images of the target from the 3D image data at step 116. Enface images include, e.g., a target enface image that images a target in the eye and a retinal enface image that images the retina of the eye. The retinal enface image may also show the shadow cast by the target onto the retina. The OCT device generates 3D images of the target at step 118. The 3D image 62 images a volume within the eye. A computer tracks the target using the 2D images at step 120. In the embodiments, the computer may use the enface image of the target or the target shadow to track the target.

The computer performs image processing of the images to determine features of the target at step 122. For example, the OCT device generates a target enface image that shows a target candidate and a retinal enface image that shows a shadow, and compares the target candidate and the shadow to confirm the presence of the target. As another example, a computer program may identify an outline of the target from the target enface image, and determine the size and/or shape of the target from the outline. As another example, a computer program may detect the darkness of the target shadow from the retinal enface image, and determine the density or thickness of the target from the darkness of the shadow.

The computer generates image overlays at step 124 to superimpose over images of the eye. For example, the computer may superimpose an outline of the target onto a retinal enface image, a no-fire zone onto a target enface image, or the target size, shape, and/or density onto a real time video of the eye. The computer displays the images at step 126. The laser device directs a laser beam towards the target via the same xy-scanner at step 130. OCT device 22 and laser device 20 share xy-scanner 24, allowing for co-registration between the imaging and treatment beams for more precise aiming of the laser beam. The method then ends.

A component (such as the control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic surgical system for imaging a target in an eye, comprising:
an optical coherence tomography (OCT) device configured to:
direct an imaging beam along an imaging beam path towards the eye, wherein:
an eye axis of the eye defines a z-axis within the eye; and
the z-axis defines a plurality of xy-planes within the eye;
receive the imaging beam reflected from the eye;
generate three-dimensional (3D) image data from an imaging beam reflected from the eye; and
generate a plurality of two-dimensional (2D) enface images from the 3D image data, wherein:
each of the plurality of 2D enface images is disposed along one of the plurality of xy-planes;
an xy scanner configured to:
receive the imaging beam from the OCT device and direct the imaging beam along the imaging beam path toward the target; and
a computer configured to:
perform image processing on the plurality of 2D enface images to determine one or more features of the target, wherein:
the one or more features comprise at least one of a size, shape, location, or density of the target.

2. The ophthalmic surgical system of claim 1, wherein the OCT device comprises at least one of:
a swept-source OCT (SS-OCT);
a line-field OCT device;
a full-field OCT device; or
a spectral-domain OCT (SD-OCT) device.

3. The ophthalmic surgical system of claim 1, wherein the xy scanner comprises at least one of:
a galvo scanner;
an electro-optical scanner; or
an acousto-optical scanner.

4. The ophthalmic surgical system of claim 1, wherein the target comprises a vitreous eye floater.

5. The ophthalmic surgical system of claim 1, wherein determining the one or more features of the target comprises:
performing the image processing on the plurality of 2D enface images to identify an outline of the target; and
determining the size or shape of the target based on the outline.

6. The ophthalmic surgical system of claim 1, wherein determining the one or more features of the target comprises:
performing the image processing on the plurality of 2D enface images to identify a shadow of the target; and
determining the location, size, or density of the target based on the shadow.

7. The ophthalmic surgical system of claim 1, wherein determining the one or more features of the target comprises:
performing the image processing on the plurality of 2D enface images to identify an outline and a shadow of the target; and
comparing the outline and the shadow to confirm the presence of the target.

8. The ophthalmic surgical system of claim 7, wherein confirming the presence of the target comprises:
comparing the outline and the shadow to determine that the target casts the shadow.

9. The ophthalmic surgical system of claim 1, wherein the computer is further configured to:
track the target based on one or more of the plurality of 2D enface images depicting the target.

10. The ophthalmic surgical system of claim 1, wherein the computer is further configured to:
track the target based on one or more of the plurality of 2D enface images depicting a shadow of the target.

11. The ophthalmic surgical system of claim 1, wherein the computer is further configured to:
generate and superimpose an overlay onto the plurality of 2D enface images; and
display the plurality of 2D enface images with the superimposed overlay.

12. The ophthalmic surgical system of claim 11, wherein the overlay comprises at least one of:
an outline of the target;
size information of the target;
shape information of the target; or
density information of the target.

13. The ophthalmic surgical system of claim 12, wherein:
the plurality of 2D enface images comprises or is part of real time video; and
the computer is configured to superimpose the overly onto the real time video.

14. A method for imaging a target in an eye, comprising:
directing, by an optical coherence tomography (OCT) device, an imaging beam along an imaging beam path towards the eye, wherein:
an eye axis of the eye defines a z-axis within the eye; and
the z-axis defines a plurality of xy-planes within the eye;
receiving, by an xy-scanner, the imaging beam from the OCT device and directing the imaging beam along the imaging beam path toward the target;
receiving, by the OCT device, the imaging beam reflected from the eye;
generating, by the OCT device, three-dimensional (3D) image data from the reflected imaging beam;
generating, by the OCT device, a plurality of two-dimensional (2D) enface images from the 3D image data, wherein:
each of the plurality of 2D enface images is disposed along one of the plurality of xy-planes; and
the plurality of 2D enface images comprises at least one target enface image depicting target in the eye and at least one retinal enface image depicting a retina of the eye, the retinal enface image showing a shadow cast by the target onto the retina; and
comparing the target of the target enface image and the shadow of the retinal enface image to confirm the presence of the target.

15. The method of claim 14, wherein generating each of the plurality of 2D enface images from the 3D image data comprises:
taking a slice of the 3D image data; and
performing at least one of summing, averaging, or projecting data of the slice to yield a 2D enface image.

16. The method of claim 14, further comprising:
performing, by the computer, image processing on the plurality of 2D enface images to determine one or more features of the target, wherein:
the one or more features comprise at least one of a size, shape, location, or density of the target.

17. An ophthalmic surgical system for imaging a target in an eye, comprising:
an optical coherence tomography (OCT) device configured to:
direct an imaging beam along an imaging beam path towards the eye, wherein:
an eye axis of the eye defines a z-axis within the eye; and
the z-axis defines a plurality of xy-planes within the eye;
receive the imaging beam reflected from the eye;
generate three-dimensional (3D) image data from an imaging beam reflected from the eye; and
generate a plurality of two-dimensional (2D) enface images from the 3D image data, wherein:
each of the plurality of 2D enface images is disposed along one of the plurality of xy-planes; and
the plurality of 2D enface images comprises at least one target enface image depicting target in the eye and at least one retinal enface image depicting a retina of the eye, the retinal enface image showing a shadow cast by the target onto the retina;
an xy scanner configured to:
receive the imaging beam from the OCT device and direct the imaging beam along the imaging beam path toward the target; and
a computer configured to:
compare the target of the target enface image and the shadow of the retinal enface image to confirm the presence of the target.

18. The ophthalmic surgical system of claim 17, wherein generating each of the plurality of 2D enface images from the 3D image data comprises:
taking a slice of the 3D image data; and
performing at least one of summing, averaging, or projecting data of the slice to yield a 2D enface image.

19. The ophthalmic surgical system of claim 17, wherein the computer is further configured to:
perform image processing on the plurality of 2D enface images to determine one or more features of the target, wherein:
the one or more features comprise at least one of a size, shape, location, or density of the target.

20. The ophthalmic surgical system of claim 17, wherein the computer is further configured to:
track the target based on one or more of the plurality of 2D enface images depicting the target or a shadow of the target.
